(19) United States
(12) Patent Application Publication
(10) Pub. No.: US 2024/0261039 A1
(43) Pub. Date: Aug. 8, 2024

(54) SYSTEM AND METHOD FOR BONE SURGERY

(71) Applicant: Think Surgical, Inc., Fremont, CA (US)

(72) Inventor: Joel Zuhars, Fremont, CA (US)

(73) Assignee: Think Surgical, Inc., Fremont, CA (US)

(21) Appl. No.: 18/432,407

(22) Filed: Feb. 5, 2024

Related U.S. Application Data
(60) Provisional application No. 63/443,445, filed on Feb. 5, 2023.

(51) Int. Cl. A61B 34/30; A61B 17/14; A61B 17/16; A61B 34/00; A61B 34/10; A61B 34/20

(52) U.S. Cl. CPC A61B 34/30; A61B 17/14; A61B 17/16; A61B 34/10; A61B 34/20; A61B 34/25; A61B 2034/105; A61B 2034/107; A61B 2034/2059; A61B 2034/252; A61B 2034/305

(57) ABSTRACT

A system is provided that includes a surgical robot having an end-effector and a plurality of actuators for moving the end-effector. The end-effector movement being in response to first control signal to align the end-effector with a first virtual plane having a first predefined location relative to a first portion of a bone or bone fragments for performing a first operation thereon. A second control signal aligns the end-effector with a second virtual plane having a second predefined location relative to a second portion of the bone or bone fragments for performing a second operation thereon. A feedback mechanism can also be provided based on relative locations. A method for repairing a defect in a bone is provided based on the operation of an inventive system to move the end-effector. Bone modification results to correct a defect in the bone.

### RELATED APPLICATIONS

This application is a non-provisional application that claims priority benefit of U.S. Provisional Application Ser. No. 63/443,445 filed Feb. 5, 2023; the contents of which are hereby incorporated by reference.

### TECHNICAL FIELD

The present invention generally relates to the field of orthopedic surgery, and more particularly to systems and methods for bone defect repair associated with malignancy or trauma.

### BACKGROUND OF THE INVENTION

Bone malignancies such as osteosarcomas, chondrosarcomas, Ewing sarcomas; and other conditions such as aneurysmal bone cysts, often must be addressed quickly to avoid metastasis and additional complications. Additionally, trauma that results in comminuted fracture, having multiple breaks, with the bone separated into more than two fragments, require complex and often ad hoc treatment. Instance of bone malignancies and comminuted fracture rarely result in full recovery of function to the patient. Some of the loss of function can be attributed to inaccurate surgical excision or positioning associated with the nature of the procedures.

By way of example, a subject with an osteosarcoma may need to undergo a surgical procedure to remove tumors located in the bone. FIG. 1 depicts a prior art circumstance of a femur 10 having a bone malignancy 12 therein that needs to be excised. The surgeon may use manual tools or navigated instruments to remove the bone containing the tumor and then replace the region with a bone graft. Unfortunately, manual tools are imprecise and may leave some of the cancer mass in the remaining bone. Navigated instruments are more accurate but the surgeon has to constantly reference a display screen or other device to align the instruments in the correct position when making the bone cuts. This conventional procedure is further complicated when a prosthesis of fragments must be aligned.

In instances of a traumatic bone injury resulting in a fracture, a surgeon has the difficult job of putting the bone fragments and/or shards back together into a predetermined (or planned) position and/or orientation (POSE) (e.g., a pre-fracture or native POSE, a biomechanically stable POSE, a pre-planned POSE) to restore the patient's limb biomechanics and to promote healing. FIG. 2 depicts a prior art fractured femur resulting in a first femoral bone fragment 14 and a second femoral bone fragment 16 split at the site of the fracture 18. Re-assembly of the bone fragments and/or shards into a predetermined POSE is a complex three-dimensional problem that is rendered even more difficult by the need to fixate the bone fragments with hardware (e.g., plates, rods, screws) in the predetermined POSE.

Thus, there exists a need for systems and methods to assist in the accurate excision of osteomalignancies from a bone and to restore the alignment of the resulting bone pieces caused by the excision. There further exists a need for systems and methods to assist in aligning bone fragments in a predetermined POSE to repair bone fractures. There further exists a need for systems and methods to assist in aligning implants (e.g., grafts) in a bone undergoing a surgical procedure associated with osteomalignancies or osteotrauma.

### SUMMARY OF THE INVENTION

A system is provided that includes a surgical robot having an end-effector and a plurality of actuators for moving the end-effector. The end-effector movement being in response to:
a first control signal to align the end-effector with a first virtual plane having a first predefined location relative to a first portion of a bone for performing a first operation on the first portion of the bone; and
a second control signal to align the end-effector with a second virtual plane having a second predefined location relative to a second portion of the bone for performing a second operation on the second portion of the bone; and
a feedback mechanism communicating a first position and orientation (POSE) of the first portion of the bone with respect to a second POSE of the second portion of the bone when the first portion of the bone is separate from the second portion of the bone.

A system is also provided that includes a surgical robot having an end-effector and a plurality of actuators for moving the end-effector. The end-effector movement being in response to:
a first control signal to align the end-effector with a first virtual plane having a first predefined location relative to a first bone fragment and a second bone fragment for performing a first operation on the first bone fragment and the second bone fragment; and
a second control signal to align the end-effector with a second virtual plane having a second predefined location relative to the first bone fragment and the second bone fragment for performing a second operation on the first bone fragment and the second bone fragment.

A method for repairing a defect in a bone is provided based on the operation of an inventive system to move the end-effector. Bone modification results.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 depicts a prior art femoral bone containing a bone malignancy in need of removal;

FIG. 2 depicts a prior art femoral bone fractured into two main bone fragments along with smaller shards at a fracture site;

FIG. 3 depicts a robotic surgical system in an operating room setting according to the present invention;

FIGS. 4A and 4B depict a 2-degree of freedom device of the robotic surgical system shown in FIG. 3, where FIG. 4A is 2-DoF device in a first operational position and FIG. 4B depicts the device of FIG. 4A in a second operational position;

FIG. 5 depicts a femoral bone containing a bone malignancy with affixed tracking arrays bounding the bone malignancy;

FIG. 6 depicts predefined locations for a first virtual plane and a second virtual plane registered to the coordinates systems of the tracking arrays of FIG. 5;

FIG. 7 depicts a device driving an end-effector, here shown as an oscillating saw, to make the cuts in the bone at the first virtual plane and successively at the second virtual plane;

FIG. 8 depicts a bone piece containing the bone malignancy removed from the bone resulting in two bone pieces each have a tracking array affixed thereto as originally installed;

FIG. 9 depicts the re-alignment of the two bone pieces of FIG. 8 with a bone graft therebetween to restore position and/or orientation (POSE) relative to the native bone,

FIG. 10 depicts a femoral bone fractured into two bone fragments, each fragment having a tracking array affixed thereto;

FIG. 11 depicts a graphical user interface displaying a predetermined (e.g., planned) POSE for re-aligning a first bone fragment model corresponding to the first bone fragment, and a second bone fragment model corresponding to the second bone fragment;

FIG. 12 depicts a first virtual plane defined at a location with respect to the 3-D bone fragment models of FIG. 11, where the bone fragment models are aligned in a predetermined (e.g., planned) POSE for re-aligning the bone fragments;

FIG. 13 depicts a second virtual plane defined at a location with respect to the 3-D bone fragments models of FIG. 11, where the bone fragment models are aligned in a predetermined (e.g., planned) POSE for re-aligning the bone fragments;

FIGS. 14A and 14B depict as sequential steps a device for insertion of pins in bone fragments coincident with the locations of the virtual planes at the pre-defined locations;

FIG. 15 depicts couplers coupled onto the pins so inserted to re-align the bone fragments in the predetermined (e.g., planned) POSE as shown in FIG. 11;

FIG. 16 depicts the bone fragments movement into their final planned POSE;

FIG. 17 depicts the installation of conventional hardware to fix the bone fragments in the planned POSE;

FIG. 18 depicts the installation of a bone graft between primary bone fragments based on the procedure depicted in FIGS. 11-16.

### DESCRIPTION OF THE INVENTION

The present invention has utility as a system and method to assist in the accurate excision of osteomalignancies from a bone and to restore the alignment of the resulting bone fragments caused by the excision. The present invention has further utility as a system and method to assist in aligning bone fragments in a predetermined POSE to repair bone fractures. By creating three-dimensional scans of the region of the bone in need of osteooncological or osteotrauma surgery, any subsequent surgery is performed more efficiently and with greater accuracy, resulting in better outcomes for the subject. The method is readily extended to guiding cutting and fixation of pins or other hardware in surgical procedure on the bone in question. The present invention is also helpful in the design or choice of a bone graft to be inserted at the situs of defect or fracture.

While the present invention is illustrated and described with respect to a malignancy or fracture of a femur, it is appreciated that the present invention is readily applicable to any bone that contains a malignancy, fracture, or other surgical procedures requiring the re-alignment of two or more separated bone pieces or fragments of the same bone. Furthermore, while the steps of individual pin placement and cutting of the bone are described with the use of a hand-held 2 degree-of-freedom device, it should be appreciated that all these steps are amenable to being performed with other computer-assisted surgical systems, computer-assisted surgical devices, or in some embodiments, conventional manual tools. For example, conventional manual tools may be aligned with a virtual plane by projecting light on the bone at the predefined location of a virtual plane registered to the bone. Visual, auditory, tactile, or haptic feedback may be provided to align the conventional tools with the predefined location of a virtual plane, or align other components (e.g., a graft) with respect to the bone at a predefined location.

The present invention will now be described with reference to the following embodiments. As is apparent by these descriptions, this invention can be embodied in different forms and should not be construed as limited to the embodiments set forth herein. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

All publications, patent applications, patents and other references mentioned herein are incorporated by reference in their entirety.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “bone data” refers to data related to one or more bones. The bone data may be determined: (i) prior to making modifications (e.g., bone cuts, insertion of a pin or screw, etc.) to one or more bones or bone pieces/fragments; and/or (ii) determined after one or more modifications have been made to a bone or bone piece/fragment. The bone data may include: the shapes of the one or more bones; the sizes of the one or more bones; angles and axes associated with the one or more bones (e.g., epicondylar axis of the femoral epicondyles, longitudinal axis of the femur, the mechanical axis of the femur); angles and axes associated with two or more bones relative to one another (e.g., the mechanical axis of the knee); anatomical landmarks associated with the one or more bones (e.g., femoral head center, knee center, ankle center, tibial tuberosity, epicondyles, most distal portion of the femoral condyles, most proximal portion of the femoral condyles); bone density data; bone microarchitecture data; and stress/loading conditions of the bone(s). By way of example, the bone data may include one or more of the following: an image data set of one or more bones (e.g., an image data set acquired via fluoroscopy, computed tomography (CT), magnetic resonance imaging (MRI), ultrasound, other x-ray modalities, laser scan, etc.); three-dimensional (3-D) bone models, which may include a virtual generic 3-D model of the bone, a physical 3-D model of the bone, a virtual patient-specific 3-D model of the bone generated from an image data set of the bone; and a set of data collected directly on the bone intra-operatively commonly used with imageless CAS devices (e.g., laser scanning the bone, painting the bone with a digitizer).

As used herein, an “end-effector” is a device or tool that interacts with the target object or material (e.g., bone, bone cement). Examples of an end-effector include, but not limited to, a saw (e.g., oscillating saw, linear saw, rotary saw), a burr, end-mill, reamer, drill bit, pin, screw, cutter, saw, laser, and a water-jet.

As used herein, the term “real-time” refers to the processing of input data within fractions of a millisecond to hundreds of milliseconds such that calculated values are available within 2 seconds of computational initiation.

As used herein, the terms “computer-assisted surgical device” and “CAS device” refer to devices used in surgical procedures that are at least in part assisted by one or more computers. Examples of CAS devices illustratively include tracked/navigated instruments and surgical robots. Examples of a surgical robot illustratively include robotic hand-held devices, serial-chain robots, bone mounted robots, parallel robots, or master-slave robots, as described in U.S. Pat. Nos. 5,086,401; 6,757,582; 7,206,626; 8,876,830; and 8,961,536; and U.S. Patent Publication No. 2013/0060278; which patents and patent application are incorporated herein by reference. The surgical robot may be active (e.g., automatic/autonomous control), semi-active (e.g. a combination of automatic and manual control), haptic (e.g., tactile, force, and/or auditory feedback), and/or provide power control (e.g., turning a robot or a part thereof on and off). It should be appreciated that the terms “robot” and “robotic” are used interchangeably herein. The terms “computer-assisted surgical system” and “CAS system” refer to a system comprising at least one CAS device and may further include additional computers, software, devices or instruments. An example of a CAS system may include: i) a CAS device and software used by the CAS device (e.g., cutting instructions, pre-operative bone data); ii) a CAS device and software used with a CAS device (e.g., surgical planning software); iii) one or more CAS devices (e.g., a surgical robot); iv) a combination of i), ii), and iii); and iv) any of the aforementioned with additional devices or software (e.g., a tracking system, tracked/navigated instruments, tracking arrays, bone pins, rongeur, an oscillating saw, a rotary drill, manual cutting guides, manual cutting blocks, manual cutting jigs, etc.). Embodiments of the present invention are particularly adapted for a CAS system comprising a hand-held robotic CAS device such as the 2-DoF device described below.

Also referenced herein is a “surgical plan”. A surgical plan is generated using planning software. The surgical plan may be generated pre-operatively, intra-operatively, or pre-operatively and then modified intra-operatively. The planning software may be used to plan the location for one more modifications to be made on a bone or bone piece/fragment based on bone data. The planning software may include various software tools and widgets for planning the surgical procedure. This may include, for example, planning: (i) locations for one or more cuts to be made relative to a 3-D bone model, which would define the locations for one or more cuts to be made on the bone or bone piece/fragment, and/or (ii) the locations on a 3-D bone model for inserting/mounting one or more implants (e.g., pins, screws, grafts, prosthesis), which may define the location of one or more virtual references (e.g., a virtual plane, virtual boundary, virtual axis) with respect to the 3-D bone model as further described below.

As used herein, the term “digitizer” refers to a device capable of measuring, collecting, recording, and/or designating the location of physical locations (e.g., points, lines, planes, boundaries, etc.) or bone structures in three-dimensional space. By way of example but not limitation, a “digitizer” may be: a “mechanical digitizer” having passive links and joints, such as the high-resolution electro-mechanical sensor arm described in U.S. Pat. No. 6,033,415 (which U.S. patent is hereby incorporated herein by reference); a non-mechanically tracked digitizer probe (e.g., optically tracked, electromagnetically tracked, acoustically tracked, and equivalents thereof) as described for example in U.S. Pat. No. 7,043,961 (which U.S. patent is hereby incorporated herein by reference); an end-effector of a robotic device; imaging devices (e.g., CT scans, fluoroscopy/x-ray, MRI); or a laser scanner.

As used herein, the term “digitizing” refers to the collecting, measuring, designating, and/or recording of physical locations or bone structures in space with a digitizer as data.

As used herein, the term “registration” refers to: the determination of the spatial relationship between two or more objects; the determining of a coordinate transformation between two or more coordinate systems associated with those objects; and/or the mapping of an object onto another object. Examples of objects routinely registered in an operating room (OR) illustratively include: CAS systems/devices; anatomy (e.g., bone); pre-procedure data (e.g., 3-D virtual bone models); a surgical plan (e.g., virtual planes defined relative to pre-operative bone data, cutting instructions defined relative to pre-operative bone data); and any external landmarks (e.g., a tracking array affixed to a bone, an anatomical landmark, a designated point/feature on a bone, etc.) associated with the bone (if such landmarks exist). Methods of registration known in the art are described in U.S. Pat. Nos. 6,033,415; 8,010,177; 8,036,441; and 8,287,522; and U.S. Patent Application Publication 2016/0338776. In particular embodiments with orthopedic procedures, the registration procedure relies on the manual collection of several points (i.e., point-to-point, point-to-surface) on the bone using a tracked digitizer where the surgeon is prompted to collect several points on the bone that are readily mapped to corresponding points or surfaces on a 3-D bone model. The points collected from the surface of a bone with the digitizer may be matched using iterative closest point (ICP) algorithms to generate a transformation matrix. The transformation matrix provides the correspondence between: i) a targets defined in a surgical plan (e.g., a pre-defined location for a virtual plane) relative to the location of the bone in the operating room (OR); and ii) at least one of: a CAS device (e.g., a tracking array affixed to the CAS device and, if needed, calibration data and/or kinematic data); a tracking array affixed to the bone; or other coordinate system (e.g., electromagnetic sensors) positioned with respect to the bone. In other embodiments, the registration is performed using image registration. Methods of image registration known in the art are described in U.S. Pat. No. 6,033,415; Joskowicz, Leo, et al. “FRACAS: a system for computer-aided image-guided long bone fracture surgery.” Computer Aided Surgery: Official Journal of the International Society for Computer Aided Surgery (ISCAS) 3.6 (1998): 271-288. In a specific embodiment, x-ray or fluoroscopy is used to register the bones with respect to an optical tracking system using techniques known in the art. Briefly, a tracking array having fiducial markers (e.g., radiopaque markers and optical tracking markers (e.g., LEDs)) are affixed to each bone piece/fragment. X-ray or fluoroscopy is then used to image the bone pieces/fragments to create 3-D models of each bone piece/fragment and to determine the location of each tracking array relative to its bone piece/fragment. This registers each 3-D model to their corresponding bone piece/fragment via their tracking array and allows a tracking system (e.g., optical tracking system) to track the bone pieces/fragments in real-time without the need for any additional x-ray or fluoroscopy images (although additional images may be acquired). The user can then plan the surgical procedure relative to the 3-D models already registered to the bone pieces/fragments.

As used herein is the term “optical communication” which refers to wireless data transfer via infrared or visible light that are described in U.S. Pat. No. 10,507,063 and assigned to the assignee of the present application.

As used herein. the term “bone malignancy” refers to any abnormal growth in a bone. Exemplary forms thereof include osteosarcomas, chondrosarcomas, Ewing sarcomas and aneurysmal bone cysts.

As used herein, the term “feedback mechanism” refers to any mechanism that communicates feedback data to a user. The feedback may be visual, audible, tactile, or haptic. Examples of feedback mechanisms include: user interfaces displayed on a display monitor (e.g., a graphical user interface (GUI)); lights (e.g., blinking lights, on/off lights, color changing lights); audible feedback (beeping noises that may change frequency, a single audible alert noise, a frequency changing noise); tactile feedback (e.g., vibrations that may or may not change in frequency or intensity); and haptic feedback (e.g., forces imposed on a user's hand).

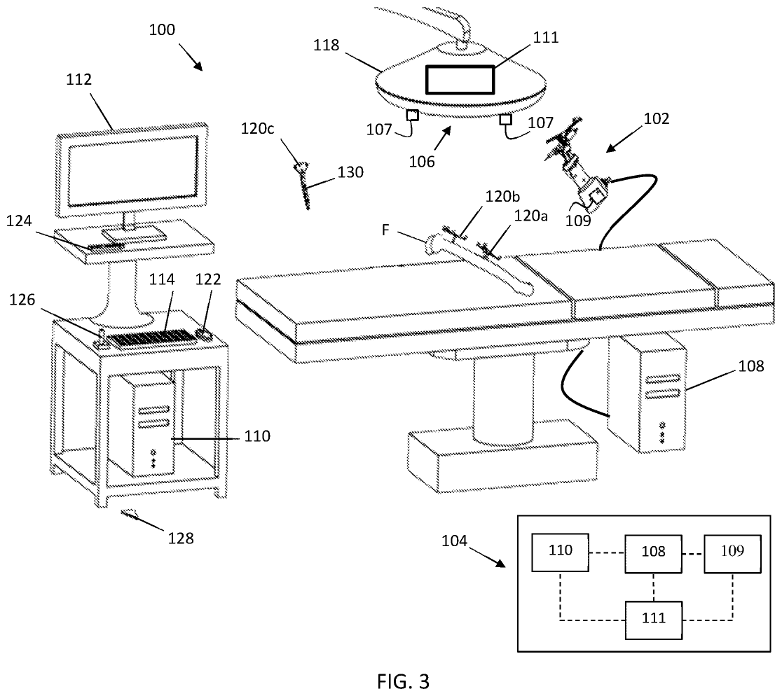

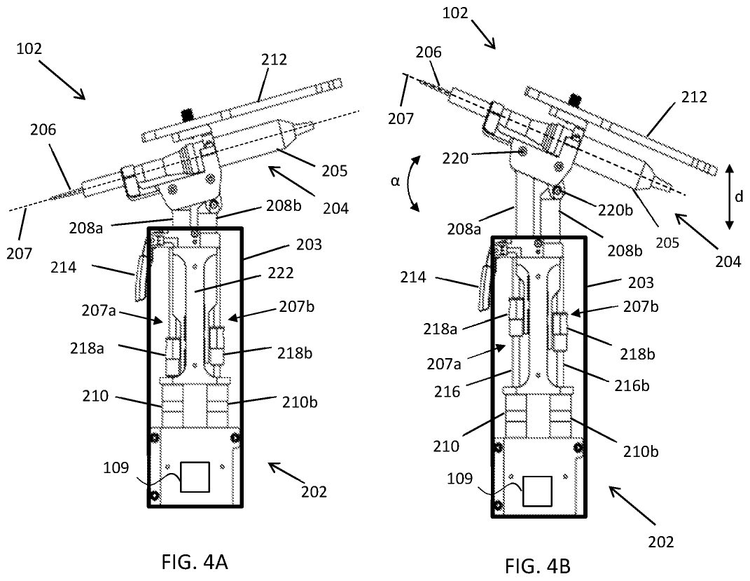

Referring to the inventive figures, FIGS. 3, 4A, and 4B, embodiments of the present inventive system and method generally includes a computer-assisted surgical system. In some inventive embodiments, a 2-DoF device 102 is provided for maintaining alignment of an end-effector coincident with a virtual plane. FIG. 3 is a schematic view showing the computer-assisted surgical system 100 including a 2-DoF device, a computing system 104, and a tracking system 106. In other inventive embodiments, an end effector extending from a robotic arm.

The computing system 104 generally includes hardware and software for executing a medical procedure. By way of example but not limitation, in one preferred form of the present invention, the computing system 104 is configured to control the actuation of the working portion 204 relative to the hand-held portion 202 of the 2-DoF 102 device to maintain alignment of the end-effector axis 207 (FIG. 2B) coincident with a virtual plane defined in a surgical plan. The working portion 204 in operation modifies subject bone. The computing system 104 may generate control signals to accurately maintain the end-effector axis 207 coincident with a virtual plane defined in the surgical plan based on: a) the location of the virtual plane registered to the location of the tissue; b) the tracked location of the tissue; and c) the tracked POSE of the 2-DoF device 102.

The computing system 104 of the computer-assisted surgical system 100 may include: one or more device computers (108, 109) including a planning computer 110; a tracking computer 111, and peripheral devices. Each computer may include one or more processors. Processors operate in the computing system 104 to perform computations and execute software associated with the inventive system and method. The device computer(s) (108, 109), the planning computer 110, and the tracking computer 111 may be separate entities as shown in FIG. 3, or it is also contemplated that operations may be executed on one (or more) computers depending on the configuration of the computer-assisted surgical system 100. It is further appreciated that one or more of the computers may be readily located remote from the surgical site. Cloud-based computation is also contemplated in the present invention. For example, the tracking computer 111 may have operational data to control the 2-DoF device 102 without the need for a device computer (108, 109). Furthermore, if desired, any combination of the device computers (108, 109), planning computer 110, and/or tracking computer 111 may be connected together via a wired or wireless connection. In addition, the data gathered by, and/or the operations performed by, the tracking computer 111 and device computer(s) (108, 109) may work together to control the 2-DoF device 102 and, as such, the data gathered by, and/or the operations performed by, the tracking computer 111 and device computer(s) (108, 109) to control the 2-DoF device 102 may be referred to herein as a “control system.”

The peripheral devices allow a user to interface with the computing system 104 and may include, but are not limited to, one or more of the following: one or more user-interfaces, such as a display or monitor (112a, 112b) to display a graphical user interface (GUI); and user-input mechanisms, such as a keyboard 114, mouse 122, pendent 124, joystick 126, and foot pedal 128. If desired, the monitor(s) (112a, 112b) may have touchscreen capabilities, and/or the 2-DoF device 102 may include one or more input mechanisms (e.g., buttons, switches, etc.). Another peripheral device may include a tracked digitizer probe 130 to assist in the registration process. Tracking arrays 120a and 120b are assembled to the digitizer probe 130 to permit the tracking system 106 to track the POSE of the digitizer probe 130 in space. The digitizer probe 130 may further include one or more user input mechanisms to provide input to the computing system 104. For example, a button on the digitizer probe 130 may allow the user to signal to the computing system 104 to digitize a point in space to assist in registering a tissue structure to a surgical plan.

The device computer(s) (108, 109) may include one or more processors, controllers, software, data, utilities, and/or storage medium(s) such as RAM, ROM or other non-volatile or volatile memory to perform functions related to the operation of the 2-DoF device 102. By way of example but not limitation, one or more of the device computers (108, 109) may include software to control the 2-DoF device 102, e.g., generate control signals to move the working portion 204 relative to the hand-held portion 202 to a targeted POSE, receive and process tracking data, control the rotational or oscillating speed of the end-effector 206 by controlling motor 205, execute registration algorithms, execute calibration routines, provide workflow instructions to the user throughout a medical procedure, as well as any other suitable software, data or utilities required to successfully perform the procedure in accordance with embodiments of the invention. In still other inventive embodiments, the device computer (108) is equipped with an alignment alert in lieu of, or in concert with the aforementioned controls. By way of example, an alignment alert includes an auditory tone, laser projection, vibration in device 102, or a combination thereof that notifies a surgeon as to a correct POSE for a tool to perform a given function. A laser light projection is well-suited to indicate a needed POSE in those instances when a conventional tool decoupled from the control system holds an end effector.

In some embodiments, the system 100 may include a first device computer 108 located separate from the 2-DoF device 102 and a second device computer 109 housed in the 2-DoF device 102 to provide on-board control. The first device computer 108 may be dedicated to the control of the surgical workflow via a GUI, the registration process and the associated calculations, the display of 3-D models and 3-D model manipulation or animation, as well as other processes. The second device computer 109, also referred to herein as an on-board device computer, may be dedicated to the control of the 2-DoF device 102. For example, the on-board device computer 109 may compute and generate the control signals for the actuator motors (210a, 210b) based on: i) received signals/data corresponding to the real-time POSE of the 2-DoF device from the tracking system; and ii) received signals/data corresponding to the real-time POSE of the virtual plane computed by first device computer 108. The on-board device computer 109 may also send internal data (e.g., operational data, actuator/screw position data, battery life, etc.) via a wired or wireless connection. In some inventive embodiments, wireless optical communication is used to send and receive the signals/data described herein. Details about bi-directional optical communication between a 2-DoF device 102 and a tracking system 106 are further described below.

The planning computer 110 in some inventive embodiments is dedicated to planning the procedure. By way of example but not limitation, the planning computer 110 may contain hardware (e.g., processors, controllers, memory, etc.), planning software, data, and/or utilities capable of: receiving, reading, and/or manipulating medical imaging data; segmenting imaging data; constructing and manipulating three-dimensional (3D) virtual models; storing and providing computer-aided design (CAD) files such as bone pin CAD files; planning the POSE of virtual planes, screws, pins, implants, grafts, and fixation hardware relative to pre-procedure data; generating the surgical planning data for use with the system 100, and providing other various functions to aid a user in planning the surgical procedure. The planning computer also contains software dedicated to defining virtual planes with regards to embodiments of the invention as further described herein. The final surgical plan data may include one or more images of target tissue or virtual models of the target tissue, tissue registration data, subject identification information, the POSE of one or more pins, screws, implants, grafts, fixation hardware relative to the tissue, and/or the POSE of one or more virtual planes defined relative to the tissue. The device computer(s) (108, 109) and the planning computer 110 may be directly connected in the operating room, or the planning computer 110 may exist as separate entities outside the operating room. The final medical plan is readily transferred to a device computer (108, 109) and/or tracking computer 111 through a wired (e.g., electrical connection) or a wireless connection (e.g., optical communication) in the operating room; or transferred via a non-transient data storage medium (e.g., a compact disc (CD), or a portable universal serial bus (USB drive)) if the planning computer 110 is located outside the operating room (or if otherwise desired). As described above, the computing system 104 may comprise one or more computers, with multiple processors capable of performing the functions of the device computer 108, the tracking computer 111, the planning computer 110, or any combination thereof.

The tracking system 106 of the present invention generally includes a detection device to determine the POSE of an object relative to the position of the detection device. In particular embodiments of the present invention, the tracking system 106 is an optical tracking system such as the optical tracking system described in U.S. Pat. No. 6,061,644 (which patent is hereby incorporated herein by reference), having two or more optical detectors 107 (e.g., cameras) for detecting the position of fiducial markers 121 arranged on rigid bodies or integrated directly on the tracked object. By way of example but not limitation, the fiducial markers 121 may include an active transmitter, such as an LED or electromagnetic radiation emitter; a passive reflector, such as a plastic sphere with a retro-reflective film; or a distinct pattern or sequence of shapes, lines or other characters. A set of fiducial markers 121 arranged on a rigid body, or integrated on a device, is sometimes referred to herein as a tracking array, where each tracking array has a unique geometry/arrangement of fiducial markers 121, or a unique transmitting wavelength/frequency (if the markers are active LEDS), such that the tracking system 106 can distinguish between each of the tracked objects.

If desired, the tracking system 106 may be incorporated into an operating room light 118, located on a boom, a stand, or built into the walls or ceilings of the operating room. The tracking system computer 111 includes tracking hardware, software, data, and/or utilities to determine the POSE of objects (e.g., tissue structures, the 2-DoF device 102) in a local or global coordinate frame. The output from the tracking system 106 (i.e., the POSE of the objects in 3-D space) is referred to herein as tracking data, where this tracking data may be readily communicated to the device computer(s) (108, 109) through a wired or wireless connection. In a particular embodiment, the tracking computer 106 processes the tracking data and provides control signals directly to the 2-DoF device 102 and/or device computer 108 based on the processed tracking data to control the position of the working portion 204 of the 2-DoF device 102 relative to the hand-held portion 202. In another embodiment, the tracking computer 106 sends tracking data to a receiver located on the 2-DoF device 102, where an on-board device computer 109 generates control signals based on the received tracking data.

The tracking data is determined in some inventive embodiments using the position of the fiducial markers detected from the optical detectors and operations/processes such as image processing, image filtering, triangulation algorithms, geometric relationship processing, registration algorithms, calibration algorithms, and coordinate transformation processing.

Bi-directional optical communication (e.g., light fidelity or Li-Fi) may occur between the 2-DoF device 102 and the tracking system 106 by way of a modulated light source (e.g., light emitting diode (LED)) and a photosensor (e.g., photodiode, camera). The 2-DoF device 102 may include an LED and a photosensor (i.e., a receiver) disposed on the working portion 204 or hand-held portion 202, where the LED and photosensor are in communication with a processor such as modem or an on-board device computer. Data generated internally by the 2-DoF device 102 may be sent to the tracking system 106 by modulating the LED, where the light signals (e.g., infrared, visible light) created by the modulation of the LED are detected by the tracking system optical detectors (e.g., cameras) or a dedicated photosensor and processed by the tracking system computer 111. The tracking system 106 may likewise send data to the 2-DoF device 102 with a modulated LED associated with the tracking system 106. Data generated by the tracking system 106 may be sent to the 2-DoF device 102 by modulating the LED on the tracking system 106, where the light signals are detected by the photosensor on the 2-DoF device 102 and processed by a processor in the 2-DoF device 102. Examples of data sent from the tracking system 106 to the 2-DoF device 102 includes operational data, medical planning data, informational data, control data, positional or tracking data, pre-procedure data, or instructional data. Examples of data sent from the 2-DoF device 102 to the tracking system 106 may include motor position data, battery life, operating status, logged data, operating parameters, warnings, or faults.

It should be appreciated that in some embodiments of the present invention, other tracking systems are incorporated with the surgical system 100. By way of example but not limitation, the surgical system 100 may comprise an electromagnetic field tracking system, ultrasound tracking systems, accelerometers and gyroscopes, and/or a mechanical tracking system. The replacement of a non-mechanical tracking system with other tracking systems will be apparent to one skilled in the art in view of the present disclosure. In one form of the present invention, the use of a mechanical tracking system may be advantageous depending on the type of surgical system used such as the computer-assisted surgical system described in U.S. Pat. No. 6,322,567 assigned to the assignee of the present application and incorporated herein by reference in its entirety.

FIGS. 4A and 4B are schematic views showing the 2-DoF device 102 in greater detail. More particularly, FIG. 4A shows the 2-DOF device 102 in a first working POSE, and FIG. 4B illustrates the 2-DOF device 102 in a second working POSE. The 2-DoF device 102 comprises a hand-held portion 202 (or handle) and a working portion 204. The hand-held portion 202 comprises an outer casing 203 of ergonomic design which can be held and wielded by a user (e.g., a surgeon). In particular embodiments, the 2-DoF device 102 is intended to be fully supported by the hands of the user in that there are no additional supporting links connected to the 2-DoF device 102 and the user supports the full weight of the 2-DoF device 102. The working portion 204 comprises an end-effector 206 having an end-effector axis 207. The end-effector 206 may be removably coupled to the working portion 204 (via a coupler (e.g., chuck)) and driven by a motor 205. The hand-held portion 202 and working portion 204 are connected to one another, for example, by a first linear actuator 207a and a second linear actuator 207b in order to control the pitch and translation of the working portion 204 relative to the hand-held portion 202, as will hereinafter be discussed in further detail. In a particular embodiment, the working portion 204 is removably coupled to the hand-held portion 202 to permit different types of working portions to be assembled to the hand-held portion 202. For example, a first working portion 204 may illustratively be a laser system having components to operate a laser for treating tissue, a second working portion 204 may illustratively be a drill for rotating a bone pin, and a third working portion 204 may illustratively be an oscillating saw.

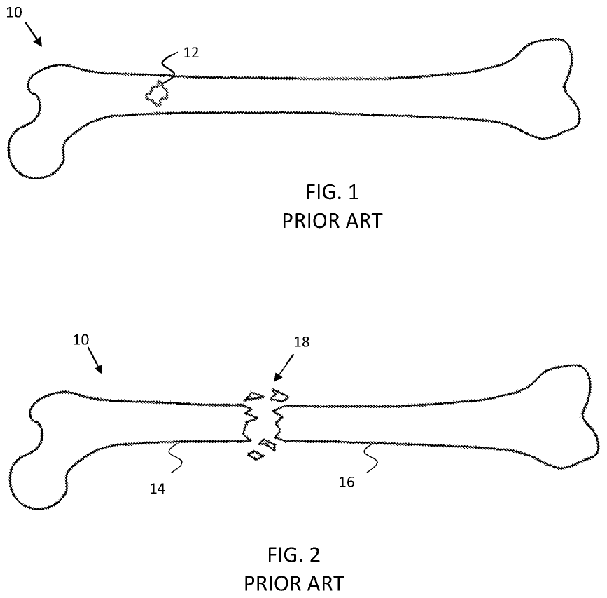

A tracking array 212, having three or more fiducial markers of the sort well known in the art, is preferably rigidly attached to the working portion 204 in order to permit the tracking system 106 (FIG. 1) to track the POSE of the working portion 204. The three or more fiducial markers may, alternatively, be integrated directly with the working portion 204. The fiducial markers may be active markers such as light emitting diodes (LEDs), or passive markers such as retroreflective spheres. The 2-DoF device 102 may further include one or more user input mechanisms such as triggers (e.g., trigger 214) or button(s). The user input mechanisms may permit the user to perform various functions illustratively including: activating or deactivating the motor 205; activating or deactivating the actuation of the working portion 204 relative to the hand-held portion 202; notifying the computing system 104 to change from targeting one virtual plane to a subsequent virtual plane; and pausing the medical procedure.

The outer casing of the hand-held portion 202 is the first linear actuator 207a and the second linear actuator 207b. Each linear actuator (207a, 207b) may include a motor (210a, 210b) to power a screw (216a, 216b) (e.g., a lead screw, a ball screw), a nut (218a, 218b), and a linear rail (208a, 208b). In some inventive embodiments, the motors (first motor 210a, second motor 210b) are electric servo-motors that bi-directionally rotate the screws (216a, 216b). Motors (210a, 210b) may also be referred to herein as linear actuator motors. The nuts (218a, 218b) (e.g, ball nuts, elongated nuts) are operatively coupled to the screws (216a, 216b) to translate along the screws (216a, 216b) as each screw is rotated by its respective motor (210a, 210b). A first end of each linear rail (208a, 208b) is coupled to a corresponding nut (216a, 216b) and the opposing end of each linear rail (208a, 208b) is coupled to the working portion 204 via hinges (220a, 220b) such that the hinges (220a, 220b) allow the working portion 204 to pivot relative to the linear rails (208a, 208b). The motors (210a, 210b) power the screws (216a, 216b) which in turn cause the nuts (218a, 218b) to translate along the axis of the screws (216a, 216b). Translation of nuts 218a, 218b along ball screws 216a, 216b, respectively, causes translation of front linear rail 208a and back linear rail 208b, respectively, whereby to permit (a) selective linear movement of working portion 204 relative to hand-held portion 202, and (b) selective pivoting of working portion 204 relative to hand-held portion 202 of 2-DoF device 102. Accordingly, the translation “d” and pitch “a” (FIG. 2B) of the working portion 204 may be adjusted depending on the position of each nut (218a, 218b) on their corresponding screw (216a, 216b). A linear guide 222 (FIG. 2A) may further constrain and guide the motion of the linear rails (208a, 208b) in the translational direction “d”. In a particular embodiment, the nuts (216a, 216b) are elongated and couple directly to the working portion 204 via the hinges (220a, 220b), in which case the linear rails (208a, 208b) are no longer a component of the linear actuators (207a, 207b). It should be appreciated that other linear actuation mechanisms/components may be used to adjust the POSE of the working portion 204 relative to the hand-held portion 202 such as linear motors, pneumatic motors, worm drives and gears, rack and pinion gears, and other arrangements of motors and transmissions.

The 2-DoF device 102 may receive power via an input/output port (e.g., from an external power source) and/or from on-board batteries (not shown).

The motors (205, 210a, 210b) of the 2-DoF device 102 may be controlled using a variety of methods. By way of example but not limitation, according to one method of the present invention, control signals may be provided via an electrical connection to an input/output port. By way of further example but not limitation, according to another method of the present invention, control signals are communicated to the 2-DoF device 102 via a wireless connection, thereby eliminating the need for electrical wiring. The wireless connection may be made via optical communication. In certain inventive embodiments, the 2-DoF device 102 includes a receiver for receiving control signals from the computing system 104 (FIG. 3). The receiver may be, for example, an input port for a wired connection (e.g., Ethernet port, serial port), a transmitter, a modem, a wireless receiver (e.g., Wi-Fi receiver, Bluetooth® receiver, a radiofrequency receiver, an optical receiver (e.g., photosensor, photodiode, camera)), or a combination thereof. The receiver may send control signals from the computing system 104 directly to the motors (205, 210a, 210b) of the 2-DoF device 102, or the receiver may be in communication with a computer (e.g., an on-board device computer 109 as further described below) that processes signals received by the receiver and then generates the control signals for the motors (205, 210a, 210b) based on the received signals.

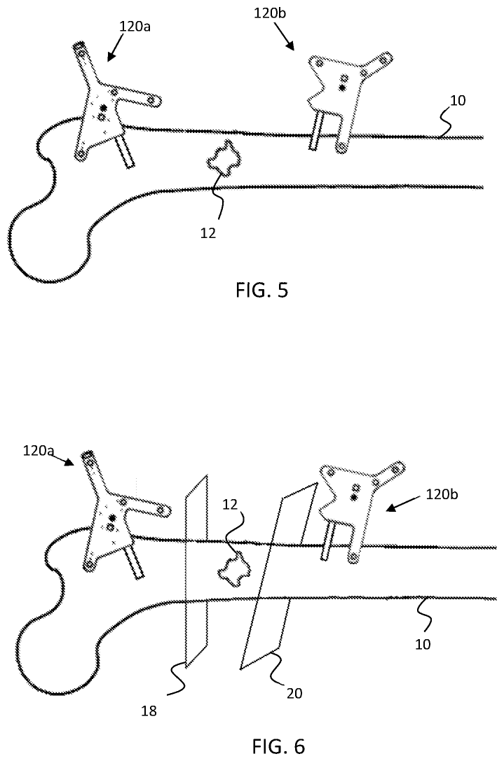

With reference now to FIG. 5, a femoral bone 10 is shown having a defect therein shown as a bone malignancy 12 in need of removal. Prior to the procedure, bone data of the femoral bone 10 is acquired (e.g., CT, x-ray, MRI) to determine the location of the tumor 12 and to determine the location of the bone cuts to be made on the bone in order to remove a bone piece containing the tumor. The planning software may define the locations of virtual planes to align coincident with respect to the predetermined (e.g., planned) locations of the bone cuts. For example, the planning software may define: (i) a location for a first virtual plane 18 (shown in FIG. 6) that aligns coincident with the location for a first cut to be made on the bone 10; and (ii) a location of a second virtual plane 20 (shown in FIG. 6) that aligns coincident with the location for a second cut to be made on the bone 10. This surgical plan may be saved, where the virtual planes are then registered to the position of the bone in the OR as further described below. In the OR, and returning back to FIG. 5, tracking arrays (120a, 120b) are affixed to the bone at locations outside the region of the bone that will be removed. For example, FIG. 5 shows a first tracking array 120a affixed to the femoral bone 10 at a location proximal to the first cut to be made on the femoral bone 10 and a second tracking array 120b affixed to the femoral bone 10 at a location distal to the second cut to be made on the femoral bone 10.

FIG. 6 depicts the predefined locations for the first virtual plane 18 and the second virtual plane 20 registered to the coordinate systems of the first tracking array 120a and the second tracking array 120b affixed to the bone 10. A 3-D bone model of the bone 10, having the predefined locations for the virtual planes (18, 20) defined with respect to the 3-D bone model, may facilitate the registration of the virtual planes (18, 20) to the coordinate systems of the tracking arrays (120a, 120b) using registration techniques known in the art. The 3-D bone model may be displayed on a GUI and move in real-time based on tracking data. It should be appreciated, that the registration process (e.g., collecting points on the bone) may only need to be performed one time in order to register the virtual planes (18, 20) to each tracking array (120a, 120b). It is appreciated that the virtual planes 18 and 20 each independently define an angle relative to the bone based on surgical preferences.

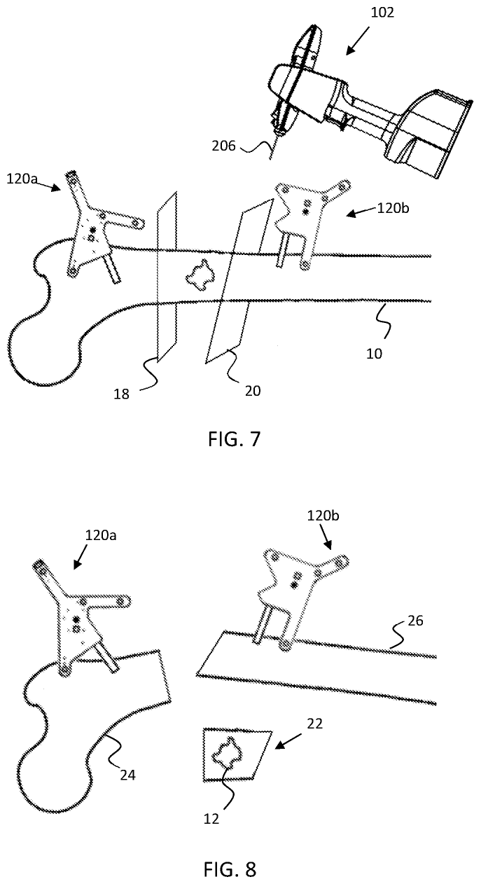

FIG. 7 depicts a surgical robot (e.g., 2-DoF device 102) driving an end-effector 206 (e.g., an oscillating saw) to make the predetermined cuts on the bone 10. It should be appreciated that end-effector 206 in addition to directly making the predetermined cuts, in other inventive embodiments places pins in subject tissue that guide a different device, such as a saw to in turn make the desired cuts. One of ordinary skill in the art will appreciate bone placement of pins is a conventional technique for the mounting of cutting jigs. The surgical robot includes a plurality of actuators for moving the end-effector 206 in response to: (i) first control signals to align the end-effector 206 with the first virtual plane 18 having the predefined location relative to the first portion of the bone (e.g., the portion of the bone on the left side of the bone malignancy 12 as shown in FIG. 7) for creating the first bone cut coincident with the first virtual plane 18; and (ii) second control signals to align the end-effector 206 with the second virtual plane 20 having the predefined location relative to the second portion of the bone (e.g., the portion of the bone on the right side of the bone malignancy 12 as show in FIG. 7) for creating a second bone cut coincident with the second virtual plane 20. The first and second control signals may be generated by a device computer (109, 110) or another computer in the computing system 104 as described above. This is a quick, precise, and accurate method for making the cuts compared to conventional methods.

In a particular embodiment, the surgical robot may align an end-effector 206 (e.g., a bone pin) to guide a different device (e.g., a conventional oscillating saw) to create the bone cuts. For example, the planning software may define: (i) a location for a first virtual plane 18 for inserting bone pins coincident with the first virtual plane; and (ii) a location for a second virtual plane 20 for inserting bone pins coincident with the second virtual plane 20. The predefined locations for the first virtual plane 18 and the second virtual plane 20 are registered to the bone using the registration techniques known in the art. The surgical robot then aligns a bone pin coincident with the predefined location of the first virtual plane for inserting two or more bone pins in the bone coincident with the first virtual plane. A cut guide, having a guide slot, is then coupled to the bone pins inserted in the bone. A conventional oscillating saw is then passed through the guide slot to create a first bone cut at a predetermined location on the first portion of the bone. The process is repeated for the second virtual plane. In this example, the orientation of the guide slot when the cut guide is coupled to the bone pins is the desired (i.e., predetermined) location for creating the bone cuts. The predefined locations for the virtual planes (i.e., the virtual planes for inserting the bone pins in the bone) may therefore be offset from the plane of the guide slot such that when the cut guide is coupled to the bone pins, the orientation of the guide slot is aligned with the desired location for making the bone cuts. An example of using a surgical robot, bone pins, and a cut guide in this manner is described in U.S. Pat. No. 11,457,980, assigned to the assignee of the present application, and incorporated by reference herein in its entirety. In another embodiment, the end-effector 206 may be a cut guide, where the surgical robot moves the cut guide to align the guide slot of the cut guide with a virtual plane, where the virtual plane corresponds to desired location for making a bone cut. Once the guide slot is aligned with the virtual plane, a conventional oscillating saw is passed through the guide slot to create the bone cut.

FIG. 8 depicts a bone piece 22 with the bone malignancy 12 removed from the bone 10, resulting in a first bone portion 24 separated from a second bone portion 25, where the first bone portion 24 and the second bone portion 26 each has its own tracking array (120a, 120b) affixed thereto as originally installed. Here, the first bone portion 24 is a first bone piece and the second bone portion 26 is a second bone piece of the same bone 10. Furthermore, while actual cuts of the bone may be coincident the in silico virtual planes 18 and 20 as shown in FIG. 8, it is appreciated that the actual cuts can be both non-linear and partially noncoincident with an associated virtual plane 18 or 20. For the purposes of describing the present invention, coincidence between a virtual plane and a bone cut is intended to include a displacement offset therebetween. By way of example a cut parallel to a virtual plane yet displaced therefrom by 5 mm is defined herein to be coincident.

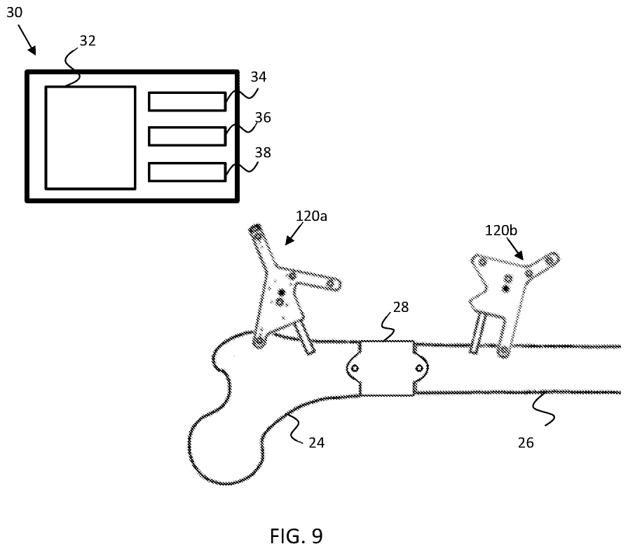

FIG. 9 depicts the re-assembly of the bone fragments (24, 26) with a bone graft 28 into their native position and/or orientation (POSE) (or other predetermined POSE), the re-alignment of which may be accomplished with tracking data and/or other feedback (e.g., a GUI 30). The bone graft 28 may be a 3-D printed component or any other bone graft known in the art (e.g., autologous bone graft, allogenic bone graft, synthetic bone graft). It is appreciated that the bone graft 28 may be customized relative to bone piece 22 or selected from a catalog of preformed articles that vary in at least dimensional or material parameter. The bone graft 28 may have a shape and/or size to mimic the shape, size, and/or function (e.g., function meaning that the graft 28 may not necessarily have the same shape or size of the removed bone piece 22 but has a shape, size, and/or mechanical properties that restores the mechanical integrity of the bone) of the removed bone piece 12. The bone pieces (24, 26) may be aligned into their native POSE using tracking data from a tracking system. In a particular embodiment, the original (i.e., pre-cut) POSE of the first tracking array 120a relative to the original POSE of the second tracking array 120b is used to re-align the bone pieces (24, 26). The user may manipulate (e.g., handle, maneuver) the bone pieces (24, 26) relative to one another while the system provides feedback to indicate when the original relative POSEs is re-established. The feedback mechanism may be visual, audio, tactile, or haptic. For example, the feedback mechanism may be a graphical user interface (GUI) 30, which may have a main window 32 that displays at least one of: (i) movement of the 3-D bone model corresponding to movement of one of the bone pieces (24 or 26) (e.g., the first bone piece 24) and the movement of the tracking array affixed to the opposing bone piece (24 or 26) (e.g., the second bone piece 26); (ii) movement of the 3-D bone model corresponding to movement of a first bone piece (24 or 26) and movement of a copy of the 3-D bone model corresponding to movement of the second bone piece (24 or 26); (iii) movement of the first tracking array 120a relative to movement of the second tracking array 120b; (iv) one or more indicators depicting the proximity and/or equality of the alignment of the first tracking array relative to the second tracking array to their original relative POSEs; and (v) any combination thereof where applicable. The GUI 30 may include other visual feedback (34, 36, 38) such as windows, indicators, signals, proximity meters, lights, graphs, targets to provide the user with an indication when the two bone pieces (24, 26) are aligned into their native POSE. When the bone pieces (24, 26) are aligned, a bone graft 28 may be coupled between the cut ends of the bone pieces (24, 26) to fixate the bone pieces (24, 26) in their native POSE to complete the procedure. In another embodiment, the method may include: (i) coupling the bone graft 28 on the end of a first bone pieces (24 or 26); (ii) aligning the bone pieces (24, 26) in their native POSE using the feedback as previously described; and (iii) once aligned, couple the bone graft 28 to the end of the opposing bone piece (24 or 26). In a further embodiment, the 2-DoF device installs pins at the end of each bone piece (24, 26) that the bone graft 28 (e.g., a 3-D printed component) couples to or references to secure the bone pieces (24, 26) in their native POSE, an example of which is described below.

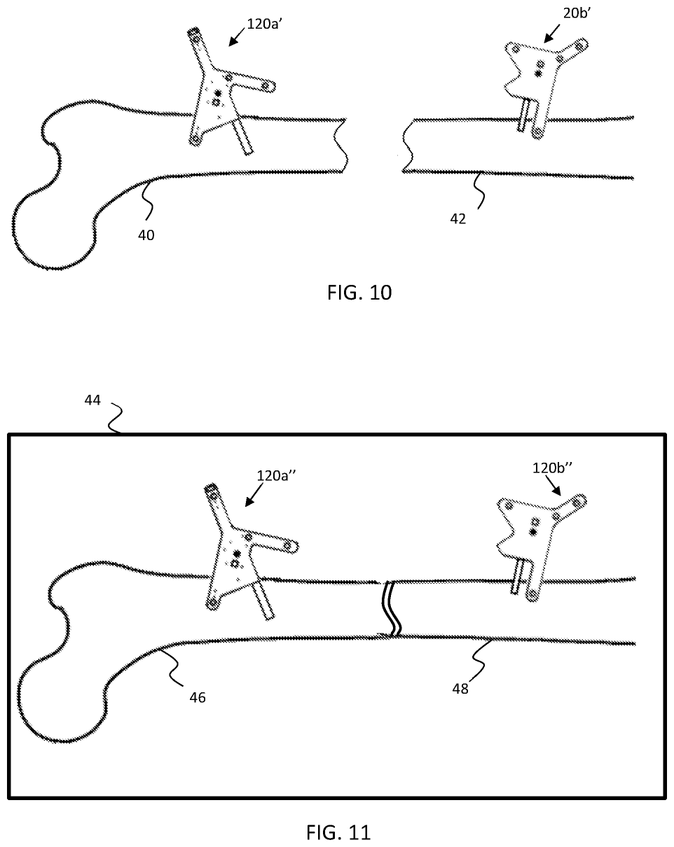

FIG. 10 depicts a defect in a femoral bone as a fracture that cleaved the bone into a first bone fragment 40 and a second bone fragment 42 as a result of a traumatic bone injury. While FIG. 10 depicts a fracture common to transverse, spiral, or compound fracture events, it is appreciated that a comminuted fracture is also readily repaired by joining primary fragments. Secondary fragments being reassembled around properly aligned primary fragments or removed and associated voids filled with cement, cadaver bone, or an implant. In the OR, a user may first affix a first tracking array 120a′ to the first bone fragment 40 and a second tracking array 120b′ to the second bone fragment 42. The first tracking array 120a′ and second tracking array 120b′ may include a plurality of fiducial markers. The fiducial markers may include at least three optical tracking fiducial markers (e.g., LEDs, passive retroreflective spheres) and at least one radiopaque fiducial marker. After each tracking array (120a′, 120b′) is affixed to its corresponding bone fragment (40, 42), images may be acquired of the bone fragments with an imaging modality (e.g., x-ray, fluoroscopy, CT, MRI, ultrasound). The images may be used to generated 3-D models of the bone fragments, also referred to herein as bone fragment model) and determine the location of each tracking array (120a′, 120b′) relative to its corresponding bone fragment (40, 42). In a particular embodiment, the at least one radiopaque fiducial marker is used to determine the location of a tracking array (120a′, 120b′) relative to its bone fragment. Then, a known geometry between the at least one radiopaque fiducial marker and the optical tracking fiducial markers may be used to provide the optical tracking system with the location of a fiducial marker array (120a, 120b) with respect to a bone fragment model (40, 42) in order to track the real-time locations of the bone fragments (40, 42) in the OR. Other methods for generating 3-D bone fragment models and determining the location of a bone fragment model with respect to a tracking array for tracking movement of the bone fragment with an optical tracking system is described in Dagnino, Giulio, et al. “Intra-operative fiducial-based CT/fluoroscope image registration framework for image-guided robot-assisted joint fracture surgery.” International journal of computer assisted radiology and surgery 12.8 (2017): 1383-1397. It is appreciated that an edge of a fracture may be splayed, sharp or otherwise amenable to revision through trimming, grinding, removal of fracture interface portions to facilitate re-assembly.

FIG. 11 depicts a graphical user interface 44 (GUI) displaying a predetermined (e.g., planned) POSE for re-aligning a first bone fragment model 46, corresponding to the first bone fragment 40, and a second bone fragment model 48, corresponding the to the second bone fragment 42. A planning computer comprising a processor, the GUI, and operating planning software may assist a user in planning the re-alignment of the bone fragments (40, 42). The planning software may be configured to generate 3-D bone fragment models (46, 48) from the image data of the anatomy, manipulate the image data and/or 3-D bone fragment models (46, 48) to designate a desired (e.g., planned, or pre-determined) POSE for re-aligning the bone fragments (40, 42), as well as other functions as described below. FIG. 11 depicts the 3-D bone fragment models (46, 48) aligned into a desired POSE for re-aligning the bone fragments (40, 42). Also shown, are the images of the tracking arrays (120a″, 120b″) and their locations relative to their 3-D bone model fragments (46, 48). The images of the tracking arrays (120a″, 120b″) may only include the radiopaque fiducials and/or radiopaque portions of the tracking arrays (120a″, 120b″) as captured by the imaging modality.

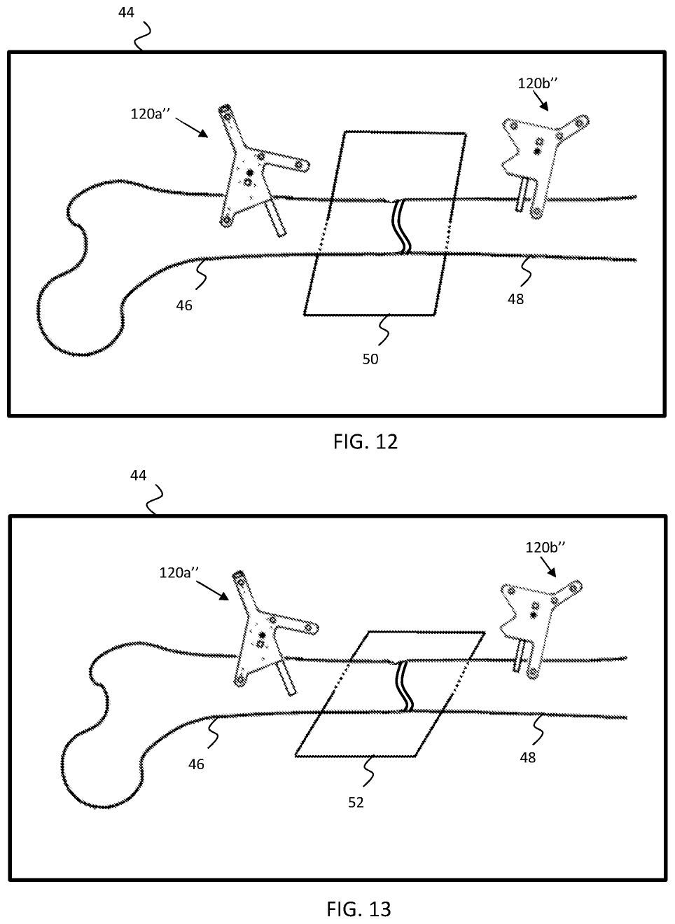

FIG. 12 depicts a first virtual plane 50 defined at a location with respect to the 3-D bone model fragments (46, 48) when the 3-D bone model fragments (46, 48) are aligned in the desired POSE for re-aligning the bone fragments (40, 42). The first virtual plane 50 intersects through at least a portion of the first bone fragment model 46 and the second bone fragment model 48. The planning software may include software tools that allow the user to define the location of the virtual planes, while in other embodiments, the planning software automatically defines (or determines) the location of the first virtual plane 50 with respect to the 3-D bone fragment models. In still other inventive embodiments, in combination with or in lieu of a GUI, a laser projection and/or auditory tone identifies a correct POSE equivalent to virtual plane 50.

FIG. 13 depicts a second virtual plane 52 defined at a location with respect to the 3-D bone model fragments (46, 48) when the 3-D bone model fragments (46, 48) are aligned in the desired POSE for re-aligning the bone fragments (40, 42). The second virtual plane 52 intersects through at least a portion of the first bone fragment model 46 and the second bone fragment model 48. The location of the second virtual plane 52 is defined as being non-parallel with the first virtual plane 50 and, in some embodiments, may be define at angle orthogonal to the first virtual plane 50. In some inventive embodiments, the intersection between planes 50 and 52 defines a line, where the line may be coaxial with the bone fragment models 50 and 52. The planning software may include software tools that allow the user to define the location of the virtual planes, while in other embodiments, the planning software automatically defines the location of the second plane 52 with respect to the 3-D bone fragment models. In still other inventive embodiments, in combination with or in lieu of a GUI, a laser projection and/or auditory tone identifies a correct POSE equivalent to the second plane 52.

In a particular embodiment, the bone fragment models 50 and 52 may be aligned into a desired POSE based on the unique geometry of the fragmented ends of each bone fragment. Since the bone fragmented in a unique way, a portion of the fragmented end on the first bone fragment will have a geometry that corresponds to a portion of the fragmented end on the second bone fragment. As such, the planning software may be used (with user input utilizing software tools, semi-automatically, or automatically) to identify the geometry of the fragmented end, or a portion thereof, of the first bone fragment model 50, identify a corresponding geometry of the fragmented end, or a portion thereof, of the second bone fragment model 52, and then match those corresponding geometries of the fragmented ends to re-align the bone fragment models (50 and 52) into their pre-fragmented POSE. With the bone fragment models (50 and 52) re-aligned according to the correspondence of the fragmented ends geometries, the first virtual plane 50 and the second virtual plane 52 may be defined as described above. By planning the POSE of the bone fragment models (50 and 52) in this manner, the bone fragments (40 and 42) will assemble together correctly and in the unique way that the bone fragmented in the first place.

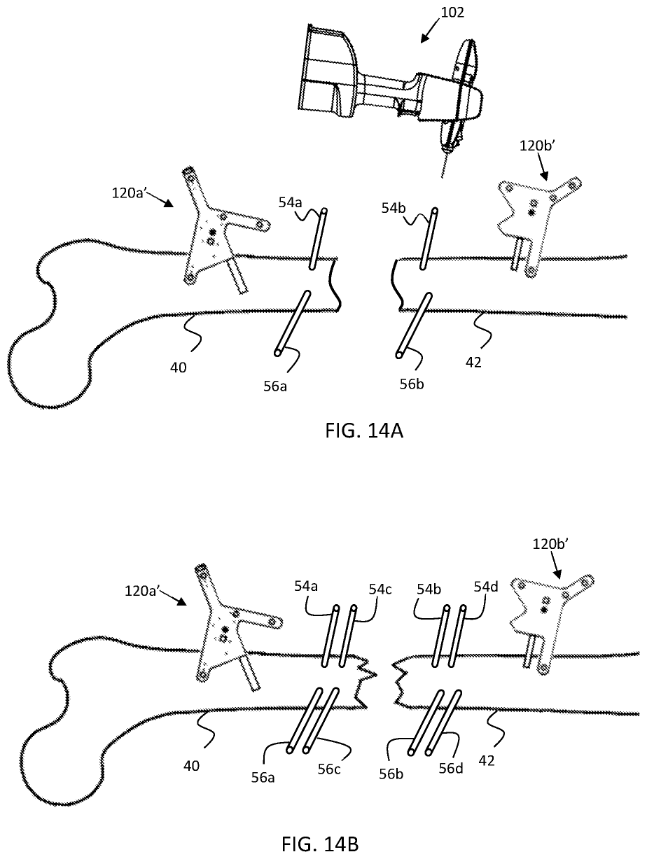

FIG. 14A depicts a surgical robot (e.g., 2-DoF device 102) for maintaining alignment of an axis of a pin (i.e., pin axis) coincident with a virtual plane (50, 52) to assist in the insertion of a plurality of pins (54a, 54b, 54c, 54d) in each bone fragment (40, 42), respectively, at locations coincident with the pre-defined locations of, or relative to, the virtual planes (50, 52). The pins may be inserted coincident with the virtual planes (50, 52) but it should be appreciated that the pins need not be placed in a virtual plane so long as the POSE of the pin relative to a given virtual plane (50, 52) is known. The predefined location for the virtual planes may be defined to avoid weak or damaged areas in a bone fragment, or the a pin may be inserted relative to the virtual plane to avoid any weak or damaged areas of a bone fragment. Since the pre-defined locations of the virtual planes (50, 52) are known with respect the imaged tracking arrays (120a′, 120b′), the pre-defined location of the virtual planes (50, 52) are also known with respect to the tracked location of the tracking arrays (120a′ 120b′) affixed to the bone fragments (40, 42). The surgical robot may therefore maintain alignment of the pin axis coincident with a virtual plane based on the following data: (i) the tracked movement of the 2-DoF device; (ii) the tracked movement of the tracking arrays (120a′, 120b′); and (iii) the known relationship between: (a) the pre-defined location of a virtual plane (50, 52) with respect to a bone fragment model (46, 48); the (b) the location of at least a portion of the imaged tracking array (120a″, 120b″) (e.g., a radiopaque fiducial marker) with respect to a bone fragment model (46, 48); and (c) any known correspondence between the imaged tracking array (120a″, 120b″) and the optical tracking fiducials of the tracking array (120a, 120b) (e.g., the known geometry of the imaged radiopaque fiducial marker with respect to the location of the optical tracking fiducial markers). The surgical robot includes a plurality of actuators to move the end-effector in response to: (i) first control signals to align the end-effector with the first virtual plane 50 having the predefined location relative to a first portion of the bone (e.g., the first bone fragment 40, or a portion of the bone comprising both the first bone fragment 40 and the second bone fragment 42 at the location of the first virtual plane 50) for inserting pins into the first portion of the bone coincident with the first virtual plane 50; and (ii) second control signals to align the end-effector with the second virtual plane 52 having the predefined location relative to a second portion of the bone (e.g., the second bone fragment 40, or a portion of the bone comprising both the first bone fragment 40 and the second bone fragment 42 at the location of the second virtual plane 52) for inserting pins into the second portion of the bone coincident with the second virtual plane 52. In a specific embodiment, a 2-DoF device 102 is used to insert a first pin 54a coincident with the first virtual plane 50 in the first bone fragment 40 and a second pin 54b coincident with the first virtual plane 52 in the second bone fragment 42. The 2-DoF device 102 is then used to insert a third pin 56c coincident with the second virtual plane 52 in the first bone fragment 40 and a fourth pin 56d coincident with the second virtual plane 52 in the second bone fragment 42.

In a particular embodiment, with reference to FIG. 14B, additional pins (54c, 54c, 56c, 56d) may be inserted into one or more bone fragments (40 and/or 42) coincident with the first virtual plane 50 and/or the second virtual plane 52. Additional pins add more coupling locations for the coupler. For example, two pins (54a, 54c) inserted coincident with the first virtual plane 50 in the first bone fragment 40 and two pins (54b, 54d) inserted coincident with the first virtual plane 50 in the second bone fragment 45 provides a total of four coupling locations for a coupler, while two pins (54a, 54c) inserted coincident with the first virtual plane 50 in the first bone fragment 40 and one pin 54b inserted coincident with the second virtual plane 52 in the second bone fragment 42 provides a total of three coupling locations for the coupler. It should be appreciated that any number and combination of pins may be inserted into the one or more bone fragments. The use of additional pins inserted into one or more bone fragments provides further stability between the bone fragments, especially in the rotational degrees-of-freedom, when the coupler is coupled to the additional pins.

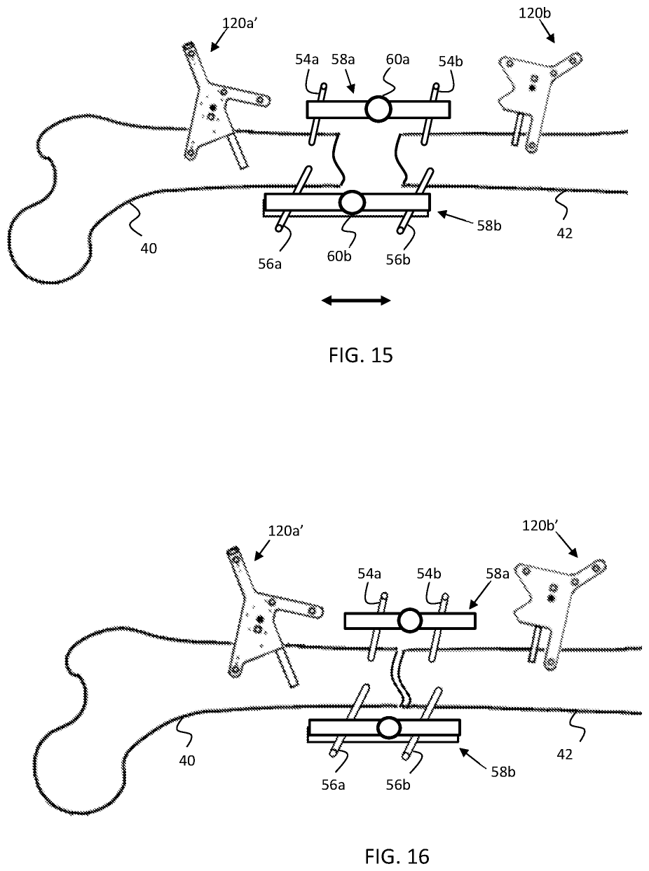

FIG. 15 depicts couplers (58a, 58b) (e.g., clamps) coupled onto the pins (56a-56d) to re-align the bone fragments (40, 42) in the planned POSE as shown in FIG. 11. In some inventive embodiments, a pin pair (54a-54b or 56a-56b) is rendered substantially parallel through forces of a coupler simultaneously engaging the pin pair. It is appreciated that more than two pins are tensioned to move into a desired orientation and thereby re-orient the bone fragment or shard in which a pin is affixed with a single coupler or several couplers. Each coupler (58a, 58b) is coupled to two pins that were aligned with, or of a known POSE relative to, the same virtual plane (50 or 52). For example, a first coupler 58a is coupled to the first pin 54a and second pin 54b present in the first bone fragment 40 and the second bone fragment 42 coincident with, or of known POSE relative to, the first virtual plane 50. A second coupler 58b is coupled to the third pin 56a and fourth pin 56b that were inserted in the first bone fragment 40 and the second bone fragment 42 coincident with, or of known POSE relative to, the second virtual plane 52. The couplers (58a, 58b) may include a pair of plates that move relative to one another using a clamping mechanism (60a, 60b) (e.g., a knob, screws, cams) to tighten and loosen the plates relative to the pins. In particular embodiments, the couplers (58a, 58b) may be lightly clamped onto their corresponding pins initially such that the bone fragments (40, 42) are brought together somewhat away from each other, but allowing the pins (and therefore the bone fragments (40, 42) to slide (or translate as shown by the arrow in FIG. 15), along the virtual planes (50, 52). The coupler may be set up to allow only a single sliding degree of freedom, such that by sliding the bones together along this path the bones will slide together correctly in the predetermined (e.g., planned) POSE. The bones may also slide together uniquely if the predetermined POSE for the bone fragment models was determined using the corresponding geometries of the fragmented ends of each bone fragment model. In instances where a graft is implanted between the bone fragments, the sliding of the bone fragments together along this path effectively sandwiches the graft between the bone fragments. The graft may be manufactured to fit uniquely (or in a single POSE) between the bone fragments, which would allow the user to quickly manipulate the graft by hand to position the graft in the correct POSE between the bone fragments.

FIG. 16 depicts the bone fragments (40, 42) moving (e.g., sliding) into the final POSE as predetermined during the planning phase of the procedure. In some embodiments, feedback (e.g., audio, visual feedback on a GUI) may be provided to signal when the bone fragments (40, 42) are at the predetermined distance from one another. However, this feedback is not specifically required because it may not be needed in some clinical cases. Once positioned in the final POSE, the couplers (58a, 58b) are tightened onto the pins using the clamping mechanism (60a, 60b). It is appreciated that in some inventive embodiments in which the pins (56a-56d), clamping mechanism (60a, 60b), or a combination thereof are dimensionally appropriate, these are used to fix the bone fragments by securing the clamping mechanisms in place.

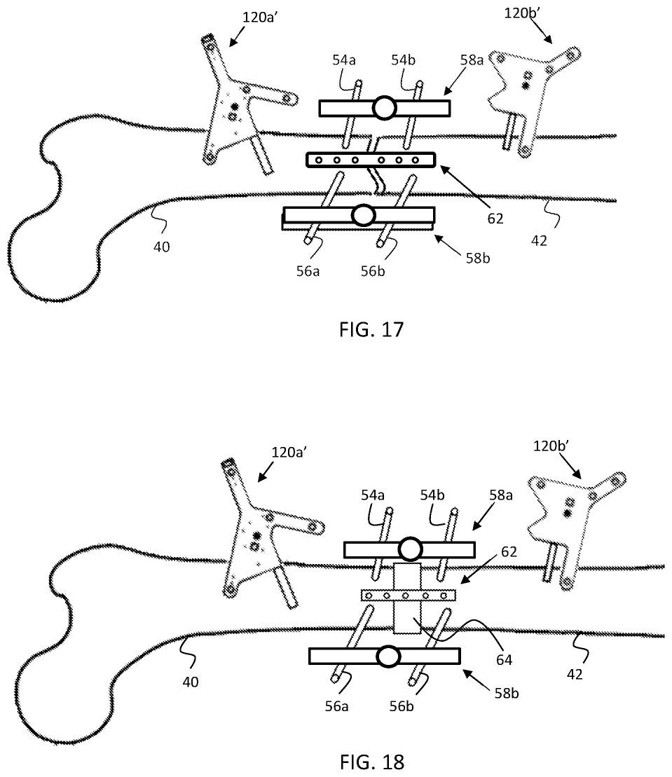

FIG. 17 depicts the installation of traditional hardware 62 (e.g., plates, screws) to fix the bone fragments (40, 42) in the predetermined POSE. After the bone fragments are fixed, the pins (54, 56), couplers 58, and tracking arrays 120 in all, or part, are removed from the bone fragments (40, 42) to complete the procedure.

In particular embodiments, the edges or ends of the bone fragments (40, 42) may be “cleaned”, modified, or revised by cutting, grinding, shaving, or sanding to form modified ends of the bone fragments (40, 42). With reference to FIG. 18, the ends of the bone fragments may be modified to correspond to the graft ends of a bone graft 64, or the graft ends of a custom bone graft 64 may be formed to correspond the modified ends of the bone fragments. In this way, the coupling of a bone graft 64 to re-assemble the bone fragments is optimized for better fixation and longevity.

It should be appreciated, that the use of the virtual planes and pins may also be used to align bone portions separated during osteooncology applications as previously described with respect to FIGS. 5-9. In particular embodiments, the location of one or more virtual planes may be defined with respect to a bone model of the bone when the bone is still intact. A surgical robot (e.g., 2-DoF device 102) may assist in inserting pins in each portion of the bone (e.g., a first portion of the bone on the left side of the bone malignancy 12 as shown in FIG. 7 and a second portion of the bone on the right side of the bone malignancy 12 as shown in FIG. 7) coincident with the one or more virtual planes before the bone cuts are made. The bone cuts are then made, which may be done with the use of a surgical robot or with manual instrumentation. The result of bone cuts is a first bone portion 24 separated from a second bone portion 26 as shown in FIG. 8 but now including at least one pin in the first bone portion 24 and at least one pin the second bone portion 26, similar to pin 54a inserted in the first bone fragment 40 shown in FIG. 14 and pin 54b inserted in bone fragment 42 shown in FIG. 14. A bone graft may include one or more features (e.g., a 3-D printed feature such as a lip, ridge, hole, channel) that references the pins (e.g., pins 54a and 54b shown in FIGS. 14A and 14B, now inserted in the bone portions (24, 26) shown in FIG. 8) to re-align the bone portions (24, 26) into the pre-cut POSE since the pins were inserted in the bone 10 prior to the bone cuts.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A system, comprising:
a surgical robot comprising an end-effector and a plurality of actuators for moving the end-effector in response to:
a first control signal to align the end-effector with a first virtual plane having a first predefined location relative to a first portion of a bone for performing a first operation on the first portion of the bone; and
a second control signal to align the end-effector with a second virtual plane having a second predefined location relative to a second portion of the bone for performing a second operation on the second portion of the bone; and
a feedback mechanism communicating a first position and orientation (POSE) of the first portion of the bone with respect to a second POSE of the second portion of the bone when the first portion of the bone is separate from the second portion of the bone.

2. The system of claim 1 wherein the first control signals and the second control signals correspond, at least in part, to movement of at least one of the first portion of the bone, the second portion of the bone, and the surgical robot to maintain alignment of the end-effector with the first virtual plane and second virtual plane, respectively.

3. The system of claim 2 wherein alignment of the end-effector with at least one of the first virtual plane or the second virtual plane is coincidence therewith.

4. The system of claim 1 wherein the feedback mechanism comprises a graphical user interface (GUI).

5. The system of claim 4 wherein the GUI displays a bone model of the bone and a location of the bone model corresponding to a first location of the first portion of the bone with respect to a second location of the second portion of the bone.

6. The system of claim 1 further comprising a first tracking array configured to be affixed to the first bone portion and a second tracking configured to be affixed to the second portion of the bone.

7. The system of claim 6 wherein bone data is registered to a first coordinate system of the first tracking array and a second coordinate system of the second tracking array.

8. The system of claim 7 wherein the feedback mechanism displays: (i) the bone data; and (ii) a location of the bone data corresponding to a location of the first portion of the bone with respect to a location of the second tracking array.

9. The system of claim 7 wherein the feedback mechanism displays: (i) at least a portion of the bone data; (ii) a location of the bone data corresponding to a location of the first portion of the bone; (iii) a copy of the bone data; and (iv) a location of the copy of the bone data corresponding to a location of the second portion of the bone.

10. The system of claim 1 wherein the feedback mechanism displays the location of the first tracking array with respect to the location of the second tracking array.

11. The system of claim 1 wherein the bone comprises a defect between the first portion of the bone and the second portion of the bone.

12. The system of claim 1 wherein the first operation is a first bone cut through the first portion of the bone and the second operation is a second bone cut through the second portion of the bone to separate the first bone portion from the second portion of the bone.

13. A system, comprising:
a surgical robot comprising an end-effector and a plurality of actuators for moving the end-effector in response to:
a first control signal to align the end-effector with a first virtual plane having a first predefined location relative to a first bone fragment and a second bone fragment for performing a first operation on the first bone fragment and the second bone fragment; and
a second control signal to align the end-effector with a second virtual plane having a second predefined location relative to the first bone fragment and the second bone fragment for performing a second operation on the first bone fragment and the second bone fragment.

14. The system of claim 13 wherein the first operation comprises aligning a first pin coincident with the first virtual plane for inserting the first pin in the first bone fragment and aligning a second pin coincident with the first virtual plane for inserting the second pin in the second bone fragment in the second bone fragment, and wherein the second operation comprises aligning a third pin coincident with the second virtual plane for inserting the third pin in the first bone fragment and aligning a fourth pin coincident with the second virtual plane for inserting the fourth pin in the second bone fragment.

15. The system of claim 13 wherein a first predefined location for the first virtual plane is defined with respect to a first bone fragment model and a second bone fragment model.

16. The system of claim 15 further comprising a first coupler for coupling to the first pin and the second pin, and a second coupler for coupling to the third pin and the fourth pin.

17. The system of claim 16 wherein the first coupler and second coupler comprise a clamping mechanism.

18. The system of claim 13 further comprising a feedback mechanism communicating a predetermined distance for fixating the first bone fragment to the second bone fragment.

19. The system of claim 13 further comprising a first tracking array configured to be affixed to the first bone fragment and a second tracking configured to be affixed to the second bone fragment.

20. A method for repairing a defect in a bone comprising:
operating the system of claim 1 to modify the bone to correct the defect.
